(19) United States
(12) Patent Application Publication
(10) Pub. No.: US 2026/0128180 A1
(43) Pub. Date: May 7, 2026

(54) HEALTH PLATFORM TRUST INDEX SYSTEM

(71) Applicant: CITYBLOCK HEALTH, INC., Brooklyn, NY (US)

(72) Inventors: Aarthi RAO, Concord, MA (US); Oluwatoyin AJAYI, Brooklyn, NY (US); Matthew VAN DEREN, Burien, WA (US)

(21) Appl. No.: 19/378,834

(22) Filed: Nov. 4, 2025

Related U.S. Application Data
(60) Provisional application No. 63/716,125, filed on Nov. 4, 2024.

(51) Int. Cl. G16H 50/70; G16H 10/20

(52) U.S. Cl. CPC G16H 50/70; G16H 10/20

(57) ABSTRACT

An example computer-implemented method described herein includes receiving a patient interaction notification for a patient from a healthcare provider for the patient, transmitting patient survey questions to a user device associated with the patient. The patient survey questions may be based on a patient's experience with the healthcare provider during the health event and may comprise benchmark categories related to the healthcare experience. The method further includes weighting the patient survey responses received based on the benchmark categories mapped to the questions and determining a trust index value for the patient using the weighted patient survey responses. The method may further include recommending an action to be performed by the healthcare provider based on the trust index value of a patient, such as patient outreach if the patient's trust index value is below a threshold value. Patient outreach may be initiated automatically by the healthcare provider software.

### CROSS-REFERENCE TO RELATED APPLICATION

This application claims the benefit of priority pursuant to 35 U.S.C. § 119(e) of U.S. Provisional Patent Application No. 63/716,125, Nov. 11, 2024, entitled “Health Platform Trust Index System,” which is hereby incorporated by reference herein in its entirety.

### TECHNICAL FIELD

The present technology is generally directed to providing individualized healthcare and more specifically to systems and methods for dynamically updating healthcare to improve patient outcomes.

### BACKGROUND

There are high levels of mistrust in healthcare. Often primary care providers have trust individually, but generally healthcare as a whole has low trust. For example, many people are skeptical of healthcare as an industry. Building trust with patients may change this overall view of health care systems and reduce skepticism. There is a gap in the healthcare industry as to how to understand what trust is, how to accurately measure it, and how to use it to drive systemic change. Further, many healthcare systems, such as scheduling platforms, patient health record systems, and the like, may further negatively impact patient trust.

There may be benefits from increasing trust in the healthcare system. Increased trust may result in patients being more likely to follow medical advice, such as following a diabetes regimen, and more likely to share health-related information, such as emergency room discharges. As a result, the cost of health care may be lowered. Various health services software platforms, such as scheduling platforms, outreach tools, and the like, do not take into account patient trust, reducing patient engagement and results.

### SUMMARY

In one embodiment, a computer implemented method is disclosed. The method includes receiving a patient interaction notification for a patient from a healthcare provider for the patient; transmitting a plurality of patient survey questions to a user device associated with the patient, wherein the plurality of patient survey questions based on a patient experience with the healthcare provider during the health event and comprise benchmark categories related to the healthcare experience; receiving patient survey responses from the user device for the plurality of survey questions; weighting the patient survey responses based on the benchmark categories; determining a patient trust index value for the patient using the weighted patient survey responses; and outputting the patient trust index value to a provider device.

In another example, the computer implemented method includes transmitting automatically an action notification to the healthcare provider based on the trust index value. For example, the action notification may recommend that a follow-up notification be transmitted to the patient because the trust index value associated with the patient is below a threshold value. The follow-up notification may be a text message, an email with additional survey questions, and/or a telephone call from a care team member to identify ways to improve the patient's experience.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments and implementations and illustrated in the accompanying drawings.

### BRIEF DESCRIPTION OF DRAWINGS

FIG. 1 illustrates an example system including a trust index system in accordance with various embodiments of the disclosure.

FIG. 2 illustrates an example process for generation of a trust index value for a patient of a healthcare provider.

FIG. 3 depicts an example patient survey landing page as displayed on a patient device according to some embodiments of the disclosure.

FIG. 4 depicts an example patient survey question page as displayed on a patient device according to some embodiments of the disclosure.

FIG. 5 depicts an example patient survey question page as displayed on a patient device according to some embodiments of the disclosure.

FIG. 6 depicts an example patient survey question page as displayed on a patient device according to some embodiments of the disclosure.

FIG. 7 depicts an example patient survey question page as displayed on a patient device according to some embodiments of the disclosure.

FIG. 8 depicts an example patient survey question page as displayed on a patient device according to some embodiments of the disclosure.

FIG. 9 depicts an example patient survey question page as displayed on a patient device according to some embodiments of the disclosure.

FIG. 10 depicts an example patient survey question page as displayed on a patient device according to some embodiments of the disclosure.

FIG. 11 illustrates an example computing system that may be used for implementing various embodiments of the disclosure.

### DETAILED DESCRIPTION

By accounting for patient trust, healthcare services software used by healthcare providers, such as scheduling platforms and outreach tools, can improve patient engagement and treatment results. When patients trust their healthcare providers and the healthcare institution, they are more likely to communicate fully with their healthcare providers; follow recommended healthcare regimes, such as a diabetes treatment regime, prescription regimens, and/or mental health treatment; and/or share otherwise unavailable health records, such as specific emergency room discharge information. Further, they are more likely to seek preventative care from their healthcare providers than to wait for problems to fester and/or escalate. This patient behavior ultimately drives down the cost of healthcare, while also improving patient outcomes. Further, many health platforms, such as scheduling tools, telehealth tools, and the like, do not track patient information or communicate across different providers, nor communicate patient data that may be helpful in providing care.

Measuring and tracking patient trust, and communicating patient data related to patient trust, such as through a metric through the healthcare services software can aid in improving the software and help to provide better healthcare services (e.g., data transfer between different providers and the patient can help to ensure that the patient profile is received before care and can be used to enhance the services provided). Patient trust spans many aspects of the healthcare system. Patients may trust their individual healthcare providers or care team members, such as individual doctors or nurses, but they may distrust the healthcare provider as an institution.

Patient surveys can be a powerful tool in measuring and tracking patient trust as a metric. There are, however, several challenges and technical problems related to tracking and evaluating trust. For example, conventional healthcare systems lack comprehensive mechanisms to systematically measure and track patient trust across different aspects of healthcare delivery, including individual provider trust, institutional trust, and trust in specific types of healthcare services. Furthermore, existing patient feedback systems typically employ generic survey approaches that fail to account for patient-specific historical data, healthcare interaction types, or institutional characteristics, resulting in suboptimal assessment of patient trust levels. Additionally, current healthcare platforms often operate in isolation without historical information for patients (both individual patients and groups of patients) and/or cross-provider information (for both individual patients and groups of patients), preventing the aggregation and analysis of patient trust data across the healthcare spectrum. The lack of automated weighting and scoring mechanisms based on benchmark categories further limits the ability to generate actionable trust metrics. Moreover, as a result, existing systems cannot prove dynamic, context-informed recommendations or implement automated interventions based on trust assessments. As a further result, existing healthcare systems frequently miss opportunities to proactively address trust issues that could improve patient outcomes and reduce healthcare costs. These unresolved issues and technical problems underscore the pressing demand for an integrated trust index system that can systematically measure, analyze, and respond to patient trust in healthcare settings.

Survey questions can be mapped to benchmarks that measure patient trust across different sectors. For example, benchmarks may include patient satisfaction, individual healthcare provider trust, institutional trust, and/or post-appointment new patient satisfaction. Each benchmark provides data on a different facet of patient trust. Patient surveys may be distributed electronically using the healthcare services software such that the benchmark data may be collected and used to determine one or more trust index values associated with the patient (e.g., trust index values may be associate with a patient profile). The trust index values may be associated with the patient's institutional trust and/or their trust in their individual healthcare provider. The trust index values may be determined by mapping the patient survey questions to benchmarks and assigning weights to the patient's responses according to the benchmark. For example, each benchmark may be assigned a percentage out of 100%, where 100% equate to complete trust. Some benchmarks may be considered more important to measuring a patient's trust and may be weighted more heavily than others. These benchmarks may change over time or by different institutions, procedures, platforms, and/or patients.

Further, the trust index values may be aggregated across different segments to evaluate patient trust as a whole for those segments, e.g., a patient cohort or section. The segments may include different characteristics associated with patients (e.g., patients in a particular location, similar economic conditions, similar health conditions, or the like) and stored in a database with patient information. For example, a trust index value may be calculated for patients in a particular geographic region, for a particular demographic of patients, for all patients with a particular type of healthcare plan, and/or for patients with a high or low risk of readmission.

The calculated trust index values may be used to determine subsequent actions to be taken by the healthcare provider and/or the healthcare services software. For example, a follow up communication may be sent automatically to patients whose trust index value is below a threshold value. A healthcare provider may also be prompted to follow up with a patient based on the patient's trust index value.

The trust index values may be input into a trust index model trained to output a recommended action for a healthcare provider to take based on the trust index values and/or various other contextual and/or historical information. For example, the trust index model may be trained to consider other factors about a patient and their situation, such as demographic information, health conditions, biological characteristics, and/or most recent healthcare provider interaction, to recommend an adjustments to operating protocols at a healthcare provider (e.g., communication techniques and/or mediums, appointment schedule times, adjustments to appointment scheduling, personnel adjustments, testing protocols, and/or the like), recommend one or more actions (e.g., follow-up communications, pro-active follow-up scheduling, refill monitoring, and/or the like), and/or prompt an automatic action performed by the healthcare services software.

There are several technical advantages of the trust index system of the present disclosure. For example, as described in more detail below, the system's ability to dynamically tailor survey questions based on patient-specific historical data, healthcare interaction types, and institutional characteristics enables more precise measurement of trust across different facets of healthcare delivery. Further, the integration of automated weighting and scoring mechanisms based on benchmark categories substantially improves the accuracy and objectivity of patient trust assessments compared to conventional generic survey approaches. Additionally, the comprehensive integration of contextual information, historical information, and/or cross-provider information allows for the systems and methods disclosed herein to account for patient trust patterns that span the entire healthcare spectrum, providing insights that isolated healthcare platforms cannot achieve. For example, as further discussed in more detail below, the integration of wide sources of information can allow the systems and methods disclosed herein to better identify small differences in trust index values. As a result, the systems and methods disclosed herein can identify meaningful differences in trust despite incomplete and/or somewhat insincere attention from patients while responding to patient survey questions, thereby helping improve an accuracy of the trust index values and/or the value of actions taken based on the trust index values. Still further, the systems and methods disclosed herein can identify actionable interventions to the healthcare process based on the trust index values to help drive increases in trust from patients. The increase in trust, in turn, can help improve outcomes for the patients overall since the patients are more likely to communicate fully with healthcare providers, follow proscribed behavior and medicine regimes, and/or seek preventive care instead of emergency and/or responsive care. These and other technical advantages discussed in more detail below collectively contribute to a comprehensive solution for healthcare trust management that enhances patient outcomes while optimizing healthcare delivery efficiency.

Various embodiments discussed in more detail below include systems to transmit patient surveys, collect the survey responses, and determine a trust index value. The healthcare system software may transmit a patient survey after receiving a notification that there was a patient interaction. The patient may interact with the healthcare provider for a variety of reasons such as medical appointments, therapy appointments, intakes by healthcare institutions, discharges from healthcare institutions, and the like. Surveys may be sent automatically using patient contact information on file. The survey questions may include visual indicators to aid in patient comprehension and accurately answering the survey questions. For example, a question asking for a rating may have color-coded options for ratings (e.g., numerical ratings of zero through ten) that change from red, indicating a poor rating, to green, indicating an excellent rating. Such visual indicators also help patients quickly answer the survey questions.

When the survey responses are received by the system, the system will determine a trust index value by weighting the responses according to the benchmark mapped to the question. The system may then be configured to recommend to the healthcare provider and/or automatically perform an action based on the trust index value. For example, the system may suggest that a healthcare provider team member follow-up with a particular patient because the patient's trust index value is below a threshold value.

In some embodiments, the trust index value may be used to determine a trust index value associated with an institution, such as a healthcare provider, or a market, such as a geographical region. For example, individual patient trust index scores for patients of a healthcare provider may be aggregated to determine the trust index value associated with the healthcare provider. Likewise, individual patient trust index scores for patients of the healthcare provider within a particular geographic area may be aggregated to determine a trust index value associated with the geographic area. In yet other embodiments, a patient engagement strategy may be based on the trust index value. For example, marketing content to be transmitted to the patient may be tailored to the patient based on the patient's trust index value.

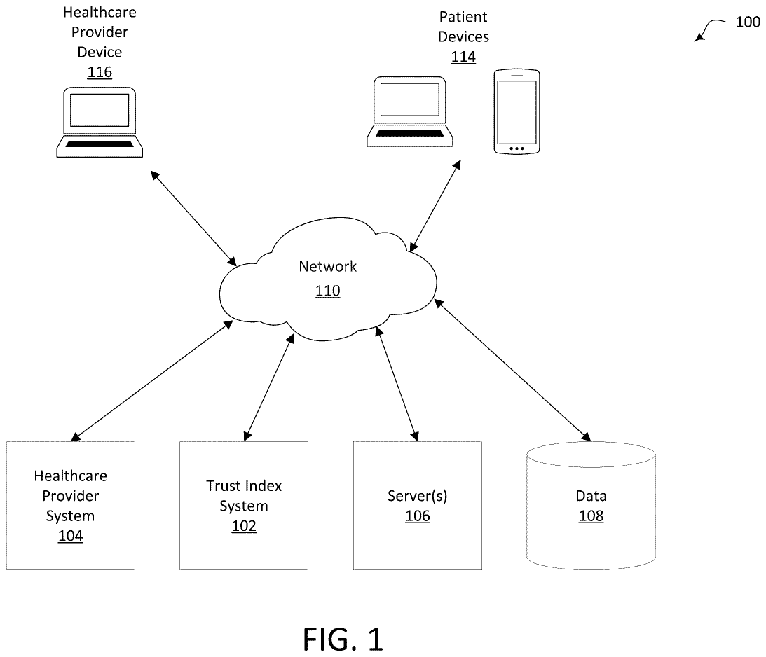

Turning now to the drawings, FIG. 1 illustrates an example system 100 including a trust index system 102 in accordance with various embodiments of the disclosure. The trust index system 102 may generally communicate with various systems, such as healthcare provider systems 104, servers 106, and various data sources, such as datastore 108, to generate a trust index value for a patient after an interaction with a healthcare provider. The interaction may be, for example, treatment for an illness or a traumatic injury, rehabilitation, psychiatric care, prenatal care, preventative care, and the like. The trust index value may indicate a level of trust a patient has for an individual healthcare provider or care team member and/or the healthcare institution as a whole. The trust index value may be transmitted to a healthcare provider device 116 to be used by care team members and/or others to implement healthcare services. In some embodiments, the healthcare provider system 104 may automatically perform an action based on the trust index value. For example, the healthcare provider system 104 may automatically transmit a follow up communication to a patient based on the patient's trust index value.

In various examples, each of the healthcare provider system 104, the servers 106, the healthcare provider device 116, and the patient devices 114 may communicate with the trust index system 102 via the network 110. The trust index system 102 may generally serve as an interface between the healthcare provider systems 104 and the patient devices 114. For example, the trust index system 102 may receive a patient interaction notification from the healthcare provider systems 104. The trust index system 102 may then transmit patient survey questions to the patient device(s) 114, e.g., through a cloud network, cellular network, etc. Responses to the patient survey questions may be received and analyzed by the trust index system 102. For example, the trust index system 102 may determine a trust index value associated with the patient based on the patient survey responses and output the trust index value to the healthcare provider system 104 and/or healthcare provider device 116.

The trust index system 102 may be generally implemented by a computing device or combinations of computing resources in various embodiments. In various examples, the trust index system 102 may be implemented by one or more servers, cloud computing resources, and/or other computing devices. The trust index system 102 may, for example, use various processing resources to communicate with various services, access data, configure user portals, create and/or use building digital assets, and the like. The trust index system 102 may further include memory and/or storage locations to store program instructions for execution by the processor and various data, for example, data used by a trust index model.

The healthcare provider system 104 may similarly be implemented by one or more computing devices or combinations of computing resources in various embodiments. Healthcare provider systems 104 may include, in various examples, systems used by hospitals, clinics, doctors' offices, rehabilitation facilities, urgent care facilities, and the like. These systems may be patient intake software, software used to create electronic health records (EHR), an admission-discharge-transfer (ADT) system, and the like. In some examples, a healthcare facility, such as a hospital, may use multiple systems to maintain patient information.

The network 110 may be implemented using one or more of various systems and protocols for communications between computing devices. In various embodiments, the network 110 or various portions of the network 110 may be implemented using the Internet, a local area network (LAN), a wide area network (WAN), cloud networking, and/or other networks. In addition to traditional data networking protocols, in some embodiments, data may be communicated according to protocols and/or standards including near field communication (NFC), Bluetooth, cellular connections, and the like.

Generally, the patient devices 114 and the healthcare provider device 116 may be devices belonging to end users, namely patients and healthcare providers. In various implementations, the patient device(s) 114 and the healthcare provider device 116 may be implemented using any number of computing devices including, but not limited to, a computer, a laptop, tablet, mobile phone, smart phone, wearable device (e.g., AR/VR headset, smart watch, smart glasses, or the like), smart speaker, vehicle (e.g., automobile), or appliance. Generally, the patient devices 114 and/or the healthcare provider device 116 may include one or more processors, such as a central processing unit (CPU) and/or graphics processing unit (GPU). The patient devices 114 and/or healthcare provider device 116 may generally perform operations by executing executable instructions (e.g., software) using the processor(s).

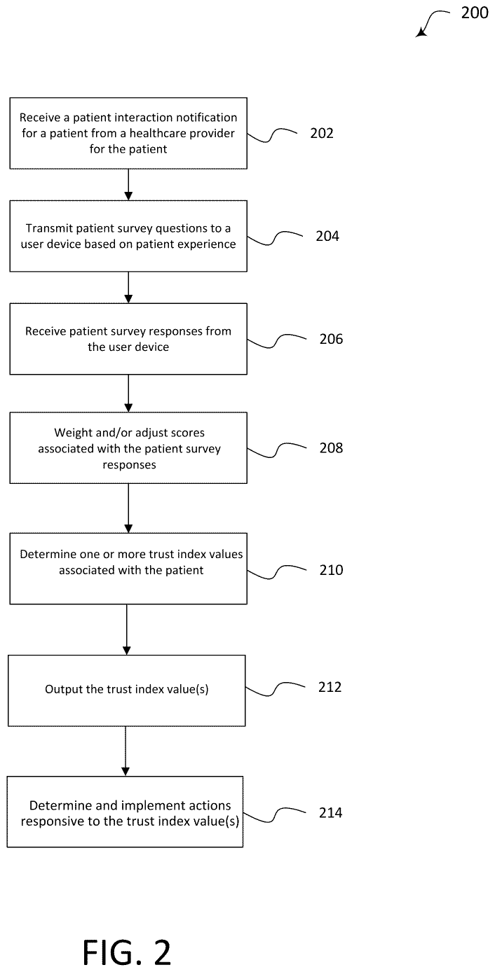

FIG. 2 illustrates an example process 200 for providing healthcare to a patient. More specifically, the process 200 may be implemented to help healthcare institutions more accurately access patient-specific trust in specific healthcare institutions (and/or healthcare overall) and/or dynamically adjust to improve patient outcomes from treatment. For example, as discussed in more detail below, the process 200 may include determining various trust index values for a patient after an interaction with a healthcare provider. In a specific, non-limiting example, the process 200 may update a patient-institution trust index value (e.g., an assessment of the patient's trust in a specific healthcare provider), a patient-healthcare trust index value (e.g., an assessment of the patient's trust in healthcare overall), and an institution trust index value (e.g., average patient trust in a specific healthcare provider). The trust index values may be based on responses to a patient survey collected by a trust index system, such as the trust index system 102 of FIG. 1, as well as historical data specific to the patient (after previous interactions with the healthcare provider and/or after interactions with other healthcare providers) and/or historical data associated with other patients (e.g., survey responses from other patients associated with the healthcare provider and/or associated with other healthcare providers). The process 200 may generate the trust index values in near-real time once the patient survey responses are received. That is, the trust index values associated with a patient and/or institution may be regularly updated and/or generated based on the most recent patient survey responses. The process 200 may then identify actions for healthcare institutions to help improve the trust. Said another way, the process 200 may allow healthcare institutions to dynamically update, tailor, and/or adjust their practices for providing and/or supporting healthcare to help build trust with their patients. The resulting improvements in trust, as discussed in more detail below, may then help improve patient outcomes (e.g., by helping ensure patients seek preventative care instead of responsive care).

At block 202, the process 200 may include detecting a patient interaction with a healthcare provider. The detection may include receiving a patient interaction notification from a healthcare provider for the patient, such as from a healthcare provider system 104 of FIG. 1. Additionally, or alternatively, the detection may include receiving a patient interaction notification from a patient user device (e.g., an app on the Patient Devices 114 of FIG. 1). In yet another example, the detection may be based on a files record request received by a first care provider (e.g., from a first healthcare provider with an established relationship with the patient, such as a primary care provider) from a second care provider (e.g., a second healthcare provider, such as a second primary care provider when the patient visits another doctor, from an emergency care provider, from a therapist, and/or the like). The patient interaction notification may be received by the trust index system (e.g., 102 of FIG. 1) by way of an ADT system. Additionally, or alternatively, the detection may include an automated detection based on various electronic records, such as a calendar for the healthcare provider, a records update at the healthcare provider related to the patient, a billing notice related to the patient, and/or the like. A patient interaction notification may be detected in response to various patient interactions with the healthcare provider. For example, the detection may occur as a patient is discharged from a healthcare facility. Patient interactions may also include intakes at healthcare facilities, appointments for treatment of an illness or injury, therapy appointments, and/or the like. In some embodiments, the healthcare provider system (e.g., 104 of FIG. 1) may be configured to automatically transmit a patient interaction notification to the trust index system (e.g., 102 of FIG. 1). In some embodiments, the patient interaction notification may be sent using a healthcare provider device (e.g., 116 of FIG. 1). In some embodiments, a patient interaction notification may be sent based on a schedule. For example, the patient interaction notification may be transmitted to the trust index system (e.g., 102 of FIG. 1) every six months for the duration of the patient's relationship with the healthcare provider. In this example, the patient interaction notification may include a record of all interactions within the previous period (e.g., each appointment with the healthcare provider, each treatment at a healthcare facility, each interaction with a therapist, any interactions with emergency services, and/or the like). It will be understood that the six-month interval is merely an example and that the patient interaction notification may be transmitted more or less frequently in accordance with the present technology.

At block 204, the process 200 may include providing a set of patient survey questions to the patient. For example, the process 200 at block 204 may include transmitting patient survey questions to the user device associated with the patient (e.g., patient devices 114 of FIG. 1). The patient survey questions may, in some examples, be sent via an electronic communication such as in an email or a text message. In some embodiments, the patient survey questions be accessible via a link to a URL provided in the electronic communication. The patient survey questions may, in some embodiments, be transmitted automatically by the healthcare provider system (e.g., 104 of FIG. 1) and/or the trust index system (e.g., 102 of FIG. 1). The patient survey questions may be stored on a server (e.g., 106 of FIG. 1) accessible by both systems. In some embodiments, the electronic communication may be a post-appointment communication. For example, a post-appointment communication email may include a link to a patient survey question landing page that prompts the patient to begin the patient survey. The patient survey questions may, in some embodiments, be presented to the patient via a user interface that prompts the patient as to how to answer. For example, the user interface may present the patient survey question and offer answers in multiple-choice form, rating form, and/or freeform text entry. The answer choices may include visual indicators to aid the patient in answering quickly and accurately. For example, a patient survey question may be “How likely are you to recommend the healthcare provider's services to others?” and the answer may be requested in the form of a rating from zero to ten, where zero indicates “not likely at all” and ten indicates “very likely.” The patient may be able to click on a number to indicate their answer. The numbers may be color coded as well. For example, zero may be red, ten may be green, and the numbers in between may be a gradient from red to green. In some embodiments, the visual indicators may be icons. For example, a smiling face may be placed next to an “excellent” rating, such as ten, and a frowning face may be placed next to a “poor” rating, such as zero. In some embodiments, there may be a combination of color coding and icons used.

The patient survey questions may include a net promotor score (“NPS”) question. Additionally, or alternatively, the patient survey questions may include informational questions (e.g., “what type of care did you receive today,” “what was the reason for your visit,” and/or the like). Additionally, or alternatively, the patient survey questions may include questions that map to one or more benchmark categories related to the healthcare experience. The benchmark categories may align with different facets of trust to be tracked by the healthcare provider. For example, benchmark categories may include patient satisfaction, individual trust with regard to a specific healthcare provider overall, individual trust with regard to one or more specific team members at a specific healthcare provider, individual trust with a type of healthcare services (e.g., trust with respect to preventative care; trust with respect to injury care; trust with respect to mental health care; trust with respect to different categories of drugs, supplements, vaccines; and/or the like), cost consciousness and/or cost satisfaction, access consciousness and/or access satisfaction, institutional trust, and/or post-appointment new patient satisfaction. In some embodiments, one or more of the patient survey questions map to individual ones of the benchmark categories. In some embodiments, one or more of the patient survey questions map to multiple ones of the benchmark categories. Additionally, or alternatively, the patient survey questions may include one or more freeform text entry prompts.

The process (e.g., via the trust index system (e.g., 102 of FIG. 1) may tailor the patient survey questions that are provided to the patient. For example, the process 200 may include dynamically selecting the patient survey questions based on the type of healthcare interaction that triggered the patient survey questions. For example, the process 200 at block 202 may select a first set of questions for a first patient that is discharged from a healthcare provider facility for the treatment for an illness or injury, and may select a second set of questions for a second patient that is discharged from an appointment for psychiatric care. The tailoring may help assess benchmark categories specific to different aspects of healthcare. By tailoring the questions specific to the healthcare interaction, the process 200 may help improve the relevance of the information gathered, thereby allowing the process 200 to more accurately assess patient trust with various aspects of their healthcare (e.g., with specific providers, specific types of healthcare, specific services, healthcare overall, and/or the like). As discussed above, the increase in accuracy may then help the process 200 better identify responsive actions to help maintain and/or improve trust, thereby helping improve outcomes for patients.

In some embodiments, the patient survey questions may be further tailored based on historical data associated with the patient. The historical data may be stored by the healthcare provider system (e.g., 104 of FIG. 1) such as in datastores (e.g., 108 of FIG. 1) and/or in any other suitable location. For example, the historical patient data may include information about prior health events and/or prior trust index values associated with the patient. The historical data may include information related to services previously received from a specific healthcare provider and/or information (e.g., trust index scores, patient responses to survey questions, anonymized healthcare data related to categories of previous treatments, and/or the like) related to services previously received from other healthcare providers. In a specific, non-limiting example, the process 200 may access one or more running trust index scores related to the patient's trust in a specific healthcare provider, trust in one or more team members at the healthcare provider, trust in various categories of healthcare, and/or institutional trust. The process 200 may then use the running trust index scores to tailor the patient survey questions. As a result, the process 200 may help increase the amount of information gained based on the patient's responses, thereby allowing the process 200 to provide a more accurate assessment of the patient's trust in various aspects of healthcare. These improvements are further enhanced via the process's access to patient-specific information from a diverse historical database (e.g., patient responses across treatment from a variety of healthcare providers).

Still further, the patient survey questions may be further tailored based on historical data associated with the specific healthcare provider and/or the historical information associated with a category of healthcare. For example, the process 200 may tailor the patient survey questions based on an average trust index score for a specific healthcare provider based on responses from a plurality of patients at the healthcare provider. In another example, the process 200 may tailor the patient survey questions based on answers to patient survey questions from other patients specific to a healthcare provider. In yet another example, the process 200 may tailor the patient survey questions based on an average trust index score for a category of healthcare based on responses from a plurality of patients at a plurality of healthcare providers. In yet another example, the process 200 may tailor the patient survey questions based on answers to patient survey questions from other patients specific to a category of healthcare. In each of these examples, similar to the discussion above, the process 200 may help increase the amount of information gained based on the patient's responses, thereby allowing the process 200 to provide a more accurate assessment of the patient's trust in various aspects of healthcare.

At block 206, the process 200 may include receiving patient survey responses. The responses may be received from the patient's user device. In some embodiments, the trust index system (e.g., 102 of FIG. 1) may automatically receive the patient survey responses when the patient submits them through the link provided at block 202. For example, the patient survey responses may be submitted through an application programming interface (API). In some embodiments, the process 200 at block 206 includes forwarding (e.g., transmitting) the responses (e.g., response data) to the healthcare provider system (e.g., 104 of FIG. 1) to be stored, for example with other data associated with the patient. In some embodiments, the process 200 at block 206 includes receiving anonymized health data from the healthcare provider, for example to help provide context (e.g., type of healthcare received, staff providing the healthcare, length of healthcare, and/or the like) to the patient survey responses. As discussed above, in some embodiments, the patient survey answers may be in the form of choices from multiple-choice questions. In some embodiments, the patient survey answers may be ratings. For example, numerical ratings, such as zero to ten where zero is a poor rating and ten is an excellent rating, or a number of stars, where zero stars is a poor rating and five stars is an excellent rating. In some embodiments, the patient survey answers may be in the form of freeform text. For example, the user interface of the patient survey questions may prompt the patient to enter any additional comments they may have to be analyzed by the trust index system (e.g., 102 of FIG. 1) for trends. In some embodiments, the patient survey answers may be in multiple different forms, such as a combination of any of the response types discussed above.

In some embodiments, the process 200 at block 206 includes assigning scores to one or more of the responses received from the patient and/or mapping the responses to one or more benchmark categories. For example, when the patient survey questions include one or more freeform text entries, the process 200 at block 206 may parse the received responses to identify one or more benchmark categories relevant to the freeform responses and/or to assign a score to the responses. In a specific non-limiting example, the process 200 may employ a natural language learning model (LLM) to understand and evaluate the patient's freeform text responses. In this specific example, the LLM may be trained on historical data and responses to identify one or more benchmark categories associated with the patient's comments and to identify a score (e.g., on a scale from 1 to 10) for each of the benchmark categories. Said another way, the process 200 may employ a specialized LLM to the freeform responses to create objective scores from the subjective comments. In some embodiments, the LLM may be trained on and/or otherwise informed by the scores that patients provide in response to one or more other patient survey questions. Said another way, the training process may use the objective scores provided in other patient survey questions to validate the LLM during the training process. Additionally, or alternatively, the LLM may use the objective scores provided in response to other patient survey questions to help inform the score that the LLM provides.

At block 208, the process 200 may include weighting and/or otherwise adjusting scores associated with the patient survey responses. The weighting and/or scoring process may be based on the benchmark categories, historical data associated with the patient, and/or historical data associated with the healthcare provider. For example, one or more of the patient survey questions may be mapped to one or more corresponding benchmark categories (e.g., patient satisfaction, institutional trust, trust with regard to a specific healthcare provider, individual trust with regard to one or more specific team members, individual trust with a type of healthcare services, cost consciousness and/or cost satisfaction, access consciousness and/or access satisfaction, and/or the like). In a specific, non-limiting example, a first patient survey question may be mapped to a first benchmark category to take a measure of the first benchmark and a second patient survey question may be mapped to the first benchmark category and a second benchmark category to take a further measure of the first benchmark and a measure of the second benchmark. The weights may be stored based on a predetermined value of each of the benchmark categories such that the process 200 (e.g., via the trust index system (e.g., 102 of FIG. 1)) may automatically apply the weights. In some embodiments, the weights are based on percentages. For example, each benchmark may be associated with a weight percentage that is less than 100%, where the sum of all the weights is 100% and 100% represents complete trust. In a specific, non-limiting example, the weighing may take the form of:

$$α A + β B + γ C , \qquad ( 1 )$$

where α, β, and γ are percentages for patient satisfaction, individual trust with regard to a specific healthcare provider team member, individual trust with regard to the institutional level, respectively; and A, B, and C are scores between 0 and 1 resulting from responses to the patient survey questions. In this example, A, B, and C may be directly scored (e.g., A=A₁*A₂* . . . . Aₙ where A₁ and so on are the scores from individual questions) or further weighted based a predetermined importance for each question to the benchmark category (e.g., A=0.4*A₁+0.6*A₂ to weight the response to A₂ more heavily than the response to A₁). The predetermined weighting scheme may help provide a repeatable measure of patient trust, especially when a patient or healthcare provider is new to the system (e.g., when there is limited historical data available). The repeatability may, in turn, help provide a more objective interpretation of the responses for new patients and/or new providers.

In other embodiments, different weight schemes may be used. In some embodiments, weights may be associated with other metrics. In some embodiments, the process 200 dynamically determines the weights assigned to the benchmark categories. For example, the process 200 may determine the weights assigned to the benchmark categories based on the type of healthcare interaction, historical data associated with the patient (specific to the healthcare provider and/or across treatment from multiple healthcare providers), historical data associated with the healthcare provider (e.g., across a plurality of patients), and/or the like. The dynamic weighting scheme may help increase an accuracy of interpretations of the information when historical benchmarks are available. Said another way, the process 200 may use the historical information to help normalize scores received from the patient via the weighting process.

In some embodiments, the process 200 dynamically determines the weights assigned to the responses based on historical information in addition to (or as an alternative to) the benchmark categories. For example, the process 200 may determine the weights assigned to the survey responses based on the type of healthcare interaction, historical data associated with the patient (specific to the healthcare provider and/or across treatment from multiple healthcare providers), historical data associated with the healthcare provider (e.g., across a plurality of patients), and/or the like. In a specific, non-limiting example, when patient that ordinarily provides a low score for one or more questions (e.g., in response to “My healthcare provider showed respect for what I had to say/what my needs/concerns were”) provides a medium or high score, that response may be weighted more heavily to value the improvement. In a related specific, non-limiting example, when the patient that ordinarily provides a low score for one or more questions provides a similar low score, the question may be weighted less heavily to reflect the average response. Similar to the discussion above, the dynamic weighting scheme may help increase an accuracy of interpretations of the information when historical benchmarks are available. Said another way, the process 200 may use the historical information to help normalize scores received from the patient via the weighting process.

At block 210, the process 200 may include determining one or more trust index values using the weighted patient survey responses. For example, the process 200 at block 210 (e.g., via the trust index system 102) may determine a first patient trust index value for the healthcare provider, a second patient trust index value for specific staff at the healthcare provider, a third patient trust index value for a healthcare category, and/or the like. Additionally, or alternatively, the process 200 may determine updates to trust index values for the healthcare provider and/or staff at the healthcare provider (e.g., updates to an average trust in the healthcare provider across patients). In some embodiments, the process 200 calculates the trust index value for the patient automatically from the weighted responses. For example, the process 200 may sum the weighted responses to arrive at the trust index values. In other embodiments, the process 200 may apply a different formula to the weighted responses to arrive at the trust index values. The trust index system (e.g., 102 of FIG. 1) may, in some embodiments, determine a trust index value for the healthcare provider as an institution by combining trust index values of individual patients of the healthcare provider. For example, the sum of the trust index values for the patients of a healthcare provider may represent the trust index value associated with the healthcare provider institution. In other embodiments, other combinations of the trust index values may be calculated.

Said another way, the addition process 200 at block 210 may account for historical data specific to the patient (e.g., the patient's average responses at a specific healthcare provider, the patient's average responses for categories of healthcare, and/or the like), historical data specific to the healthcare provider (e.g., responses from a plurality of patients at the healthcare provider), historical data specific to a category of healthcare (e.g., from a plurality of patients at a plurality of healthcare providers), and/or the like. In a specific, non-limiting example, the process 200 at block 210 may account for a patient with a low average trust index score (e.g., 20% trust) who provides a seemingly low score (e.g., 40%) that is above their average to grade the interaction more positively than directly scoring the trust, which would otherwise appear low. In another specific, non-limiting example, the process 200 at block 210 may account a patient with a high average trust index score (e.g., 95% trust) who provides a seemingly high score (e.g., 80 to grade the interaction more negatively than directly scoring the trust, which would otherwise appear high.

Additionally, or alternatively, the process 200 at block 210 may account for various other that may present in the patient response date. For example, the process 200 at block 210 may account for the category of healthcare (e.g., mental care) the patient received. That is, the additional algorithm may help normalize the trust index scores for low, medium, and/or high-trust categories of healthcare. The normalization may help add context to the trust index scores and/or help reveal otherwise minor differences between responses. Said another way, patients receiving a particular category of care may all provide relatively similar responses to the patient survey questions, despite drastic differences in the quality of care they received based on pre-existing biases for the type of healthcare. By adjusting the trust index scores to account for (e.g., normalize) the type of healthcare, the process 200 may better distinguish between good and bad experiences (as well as higher and lower resulting trusts). As a result, the process 200 may better identify adjustments to the healthcare to improve patient trust and therefore improve patient outcomes.

In some embodiments, the process 200 at block 210 adjusts and/or categorizes the trust index values in one or more segments based on the patient's information. For example, the trust index value may be categorized by a demographic characteristic associated with the patient. The demographic information may include income level, age, gender, healthcare plan, education level, ethnicity, spoken language, and/or the like. That is, the additional algorithm may help account for various aspects of the demographic information (e.g., general trends) when determining the trust index scores for. Similar to the discussion above, the demographic information may help add context to the trust index scores and/or help reveal otherwise minor differences between responses, thereby allowing the process 200 to better distinguish between good and bad experiences (as well as higher and lower resulting trusts).

In some embodiments, similar to the discussion above, the process 200 at block 210 determines one or more of the trust index values for an institution, such as the healthcare provider as a whole, or for a particular market, such as a geographic region. For example, a plurality of individual trust index values of patients may be aggregated to determine a trust index value for the healthcare provider as a whole. In some embodiments, the trust index values may be aggregated across a geographic region served by the healthcare provider. For example, the process 200 may calculate trust index values for a geographic region by aggregating the trust index values of the patients that receive healthcare from the healthcare provider in that geographic region. In some embodiments, the process 200 uses previously calculated institutional trust index values in the adjustments discussed above. Further, in some such embodiments, the individual trust index values determined above are then used to update the institutional trust index values.

At block 212, the process 200 may include outputting the trust index values. In some embodiments, the process 200 outputs the trust index values to a provider device. For example, the trust index value may be transmitted to the healthcare provider system (e.g., 104 of FIG. 1) and/or the healthcare provider device (e.g., 116 of FIG. 1). In some embodiments, the process 200 outputs the trust index values to one or more modules within a trust index system (e.g., 102 of FIG. 1) for further use. In some embodiments, the process 200 automatically sends the trust index values from the trust index system (e.g., 102 of FIG. 1) to the healthcare provider system (e.g., 104 of FIG. 1) and/or the healthcare provider device (e.g., 116 of FIG. 1). In other embodiments, the process 200 outputs the trust index values to a storage location to be retrieved by the healthcare provider system (e.g., 104 of FIG. 1) and/or the healthcare provider device (e.g., 116 of FIG. 1). One retrieved, the trust index values may be associated with the patient (as appropriate) and stored with the patient's information in the healthcare provider system (e.g., 104 of FIG. 1) as historical information for use in downstream processes (e.g., future evaluations).

At block 214, the process 200 may include determining and implementing one or more actions responsive to the trust index values. For example, the process 200 may include reviewing one or more of the trust index scores (and/or the responses to the patient survey questions, the weighting and adjustments, information related to the patient and/or treatment, and/or the like) to automatically identify adjustments to the operating protocols at the healthcare provider and/or one or more future action items.

Purely by way of example, the process 200 at block 214 may compare the patient's trust index value for a specific healthcare provider and their staff to the patient's previous trust index values and/or the patient's trust with other staff and/or other healthcare providers. In this example, the process 200 may further review information related to each treatment (e.g., the healthcare provided, staff associated with the healthcare provided, and/or the like) to identify potential causal relationships between different levels of trust (e.g., proactiveness in questions/scheduling from the healthcare providers, frequency of touchpoints, appointment lengths, demographic information associated with the healthcare providers (e.g., age, sex, native language fluency, and/or the like), and/or the like), then identify action items based on those causal explanations. Said another way, the process may identify the recommendations based on a meta-analysis for the patient-specific trust index values and/or the institution trust index values.

In a specific, non-limiting example, the process 200 could identify that a female patient has a higher level of trust when a female is involved with reproductive-related care. In this specific example, the process 200 may recommend the healthcare provider schedule a female practitioner (doctor, nurse, assistant, and/or the like) with the patient during future visits. As discussed above, the process 200 is able to use historical information to identify and magnify minor differences in trust levels. Those differences may carry forward to block 214, where the process 200 is able to use those minor differences and plurality of historical touchpoints to better identify causes of the differences in trust. That is, by referencing a wide variety of trust index scores, historical information, and related information, the process 200 may more accurately identify and help implement interventions to the healthcare. As a result, the process is better able to help improve trust from the healthcare providers and therefor better able to help improve patient outcomes.

In some embodiments, the process 200 includes transmitting an action notification to the healthcare provider based on one or more of the trust index values and/or information related to other trust index values associated with the patient (e.g., their experiences with other healthcare providers), the healthcare provider, and/or other healthcare providers. In some embodiments, the process 200 sends the action notification automatically. For example, the process 200 may transmit the action notification the healthcare provider system (e.g., 104 of FIG. 1) and/or the healthcare provider device (e.g., 116 of FIG. 1). In a specific example, the action notification may be transmitted via an API at the trust index system.

The notification may recommend one or more actions to be taken by the healthcare provider. For example, the action recommended may include sending a follow-up communication to the patient, as well as a format for the follow-up communication (e.g., based on whether the patient will trust an email, phone call, text message, and/or the like more). In some embodiments, the trust index system (e.g., 102 of FIG. 1) may transmit a follow-up to the patient automatically based on the trust index value associated with the patient. For example, if the trust index value associated with the patient is below a threshold value indicating that the patient is distrustful of at least one aspect of the healthcare institution. In another example, the action recommended may be to proactively reach out to the patient to schedule future contact points (e.g., when the trust index values indicate that the patient trusts the healthcare provider when they receive care but feels uncomfortable scheduling their appointments). In yet another example, the action recommended may include an adjustment to schedules specific to the patient and/or to the healthcare provider overall to help ensure patients trust their needs are being addressed during their appointments. In yet another example, the actions recommended may include a patient engagement strategy. For example, the marketing content sent to the patient may be tailored based on the patient's trust index value. The tailoring and transmission of the marketing content may occur automatically.

In some embodiments, the recommended action may be determined by a trust index model. For example, the trust index value output at block 212 of process 200 may be input into a trust index model and the model may output the recommended action for the healthcare provider based on the trust index value. The trust index model may be trained on data associated with patients in order to recommend an action. In some embodiments, for example, the trust index model is trained on data for patients from a plurality of healthcare institutions, thereby helping to identify trends across the institutions as well as causal differences in the treatments provided. As a result, for example, the trust index model may identify the recommendations based on the patient's data stored in the healthcare provider system (e.g., 104 of FIG. 1), as well as anonymized data associated with other patients at the healthcare provider and/or at other healthcare providers with similar attributes (e.g., similar trust index values, healthcare treatments, and/or characteristics).

In some embodiments, one or more of the trust index values and/or the implemented actions are associated with a patient profile for the patient for which the trust index value was determined. For example, the healthcare provider system (e.g., 104 of FIG. 1) may save the patient's trust index values and/or actions taken in response to the trust index values with the patient's other health information, such as in the patient's health profile and/or health records. Thus, the trust index values (and related information) may be included with the patient's health profile when the patient's health profile is shared with other healthcare institutions, such as if the patient visits a new healthcare clinic. Further, the trust index values (and related information) may be included in future analyses of the patient's trust. As a result, the process 200 may continually work to improve patient trust and therefore continually work to improve patient outcomes.

Although the blocks 202-214 of the process 200 are discussed and illustrated in a particular order, the process 200 of FIG. 2 is not so limited. In other embodiments, all or a subset of one or more of the blocks 202-214 of the process 200 may be performed in a different order and/or omitted. In these and other embodiments, all or a subset of any of the blocks 202-214 of the process 200 may be performed before, during, and/or after all or a subset of any of the other blocks 202-214 of the process 200. Purely by way of example, the determination of the trust index values at block 210 may be implemented generally simultaneously with weighting and/or adjusting the scores at block 208. In another example, the process 200 may omit block 212 altogether and instead only output recommended action items (e.g., while storing the trust index scores internally). Furthermore, a person skilled in the art will readily recognize that the process 200 may be altered and still remain within these and other embodiments of the present technology. For example, all or a subset of one or more blocks 202-214 of the process 200 may be repeated. Purely by way of example, the process 200 may repeat blocks 208-212 to calculate multiple different trust index values sequentially. The sequential calculation may be helpful, for example, when one or more of the trust index values are used in the determination of one or more other trust index values. Additionally, or alternatively, the process 200 may include various other processes to help improve the accuracy of the trust index values, and the actions determined based on the trust index values.

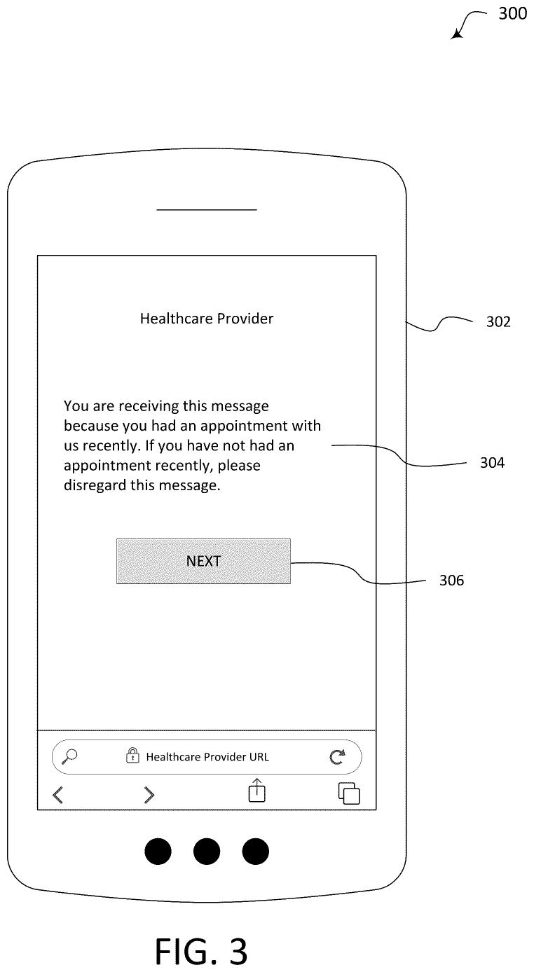

FIG. 3 depicts an example patient survey landing page 304 in a system 300 according to some embodiments of the disclosure. In some embodiments, the patient survey landing page 304 may be accessed via a link transmitted in a post-appointment communication from a healthcare provider. For example, the post-appointment communication may be transmitted by the healthcare provider system (e.g., 104 of FIG. 1) and/or the healthcare provider device (e.g., 116 of FIG. 1) in the form of a text message to be received on a patient device, such as phone 302 and/or patient devices 114 of FIG. 1. In some embodiments, the post-appointment communication may be transmitted in other forms, such as by email, post, phone call, and the like. The landing page 304 may include a message such as depicted in FIG. 3 and may prompt the patient to begin the patient survey, for example by clicking the “Next” button 306.

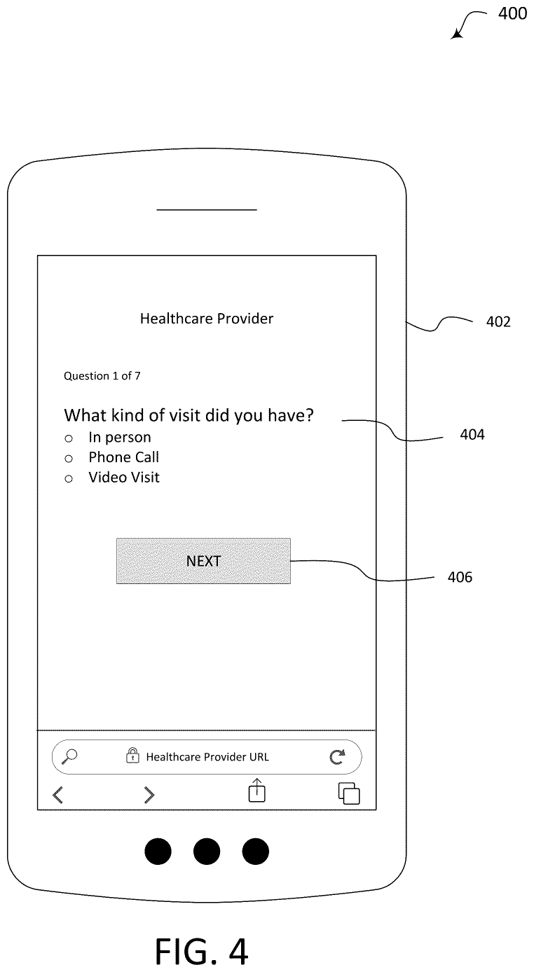

FIG. 4 depicts an example patient survey question page in a system 400 according to some embodiments of the disclosure. In some embodiments, the patient survey question page may be accessed on a patient device, such as phone 402, 302 of FIG. 3, and/or patient devices 114 of FIG. 1. The patient survey page may include one or more patient survey questions 404, of many patient survey questions, such as is depicted in FIG. 4. For example, the patient survey question page indicates that patient survey question 404 is one of seven questions. In other embodiments, there may be more or fewer patient survey questions. For example, the healthcare provider system (e.g., 104 of FIG. 1) may provide a different number of patient survey questions based on the type of patient interaction, such as treatment for an illness or a prescription refill. In some embodiments, more than one survey question may be displayed on a patient survey question page at a time. The trust index system (e.g., 102 of FIG. 1) may map the patient survey question 404 to one or more benchmark categories. In some embodiments, patient survey questions may be multiple choice, as is depicted in FIG. 4. For example, the patient may select an answer clicking or selecting on a touchscreen a radio button next to their desired response. In some embodiments, the patient may go to a next patient survey question or end the survey by clicking or selecting a button to advance, such as the “Next” button 406.

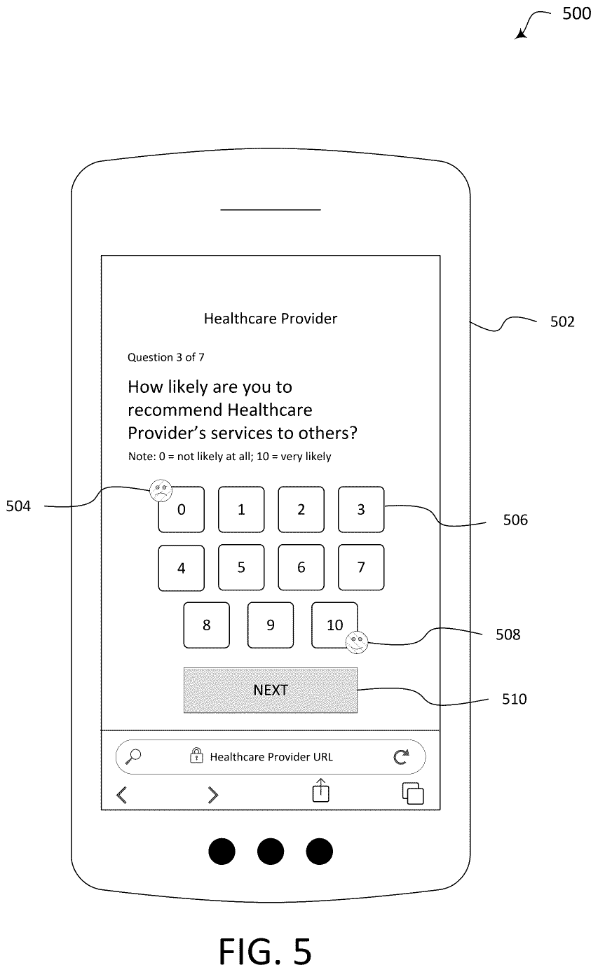

FIG. 5 depicts an example patient survey question page in a system 500 according to some embodiments of the disclosure. In some embodiments, the patient survey question page may be accessed on a patient device, such as phone 502, 402 of FIG. 4, 302 of FIG. 3, and/or patient devices 114 of FIG. 1. The patient survey page may include one or more patient survey questions, of many patient survey questions, such as depicted in FIG. 5. In some embodiments, patient survey questions may be answered on a scale, as is depicted in FIG. 5. For example, the patient may be asked to rate an experience on a scale of zero to ten, where zero means the patient's experience was poor and ten means the patient's experience was excellent. In other embodiments, zero may indicate that the patient is “not likely at all” to perform an action, such as recommend the healthcare provider to others, and ten indicates that they are “very likely” to.

In some embodiments, the scale may be depicted with visual indicators to aid in the patient's comprehension. For example, the buttons for the ratings may be colored differently to align with the rating such as the zero button may be shown in red and the ten button may be shown in green. In other embodiments, there may be other indicators such as a frowning face 504 next to the zero button and a smiling face 508 next to the ten button to correspond with the rating. In yet other embodiments, the frowning face may be shown in red, and the smiling face may be shown in green with the numbers in between shown in a gradient between red and green. The visual indicators may help the patient fill out the survey more quickly and more accurately. In some embodiments, the patient survey page may include multiple types of visual indicators. For example, the frowning face may be shown in red and the smiling face may be shown in green. The patient may go to a next patient survey question by clicking or selecting a button to advance, such as the “Next” button 510.

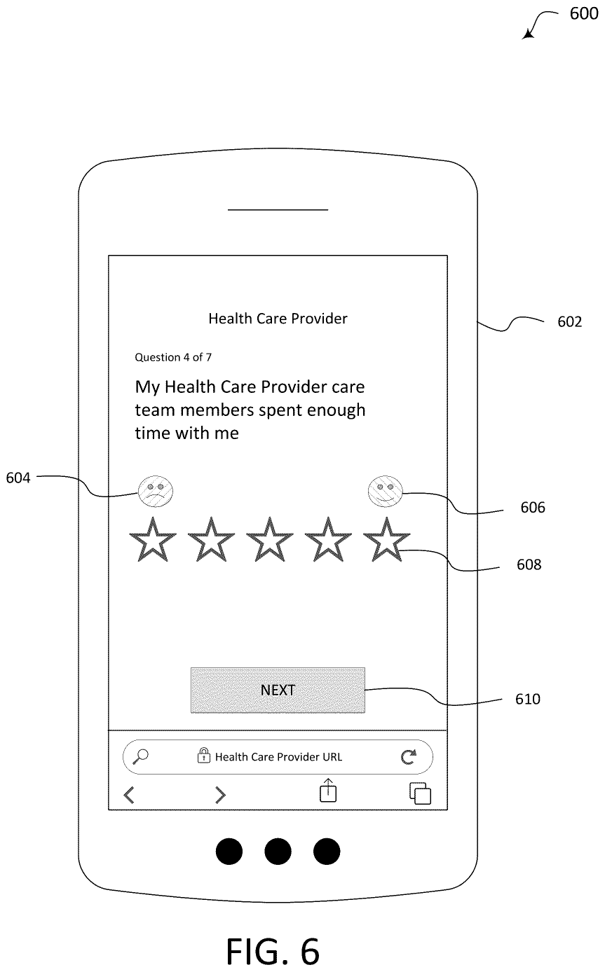

FIG. 6 depicts an example patient survey question page in a system 600 according to some embodiments of the disclosure. In some embodiments, the patient survey question page may be accessed on a patient device, such as phone 602, 502 of FIG. 5, 402 of FIG. 4, 302 of FIG. 3, and/or patient devices 114 of FIG. 1. The patient survey page may include one or more patient survey questions, of many patient survey questions, such as depicted in FIG. 6. In some embodiments, patient survey questions may be answered on a scale, as is depicted in FIG. 6. For example, the patient may be asked to rate an experience on a scale of zero “stars” to five “stars” where zero “stars” means the patient's experience was poor and five “stars” means the patient's experience was excellent. The patient may choose the number of stars by clicking on or touching a star icon so that it appears filled and the number of filled stars corresponds to the rating given by the patient. In some embodiments, the scale may be depicted in a way to aid in the patient's comprehension. For example, there may be indicators such as a frowning face next to the zero “star” button and a smiling face next to the fifth “star” button to correspond with the rating. In yet other embodiments, the frowning face 604 may be shown in red, and the smiling face 606 may be shown in green. The visual indicators may help the patient fill out the survey more quickly and more accurately. The patient may go to a next patient survey question by clicking or selecting a button to advance, such as the “Next” button 610.

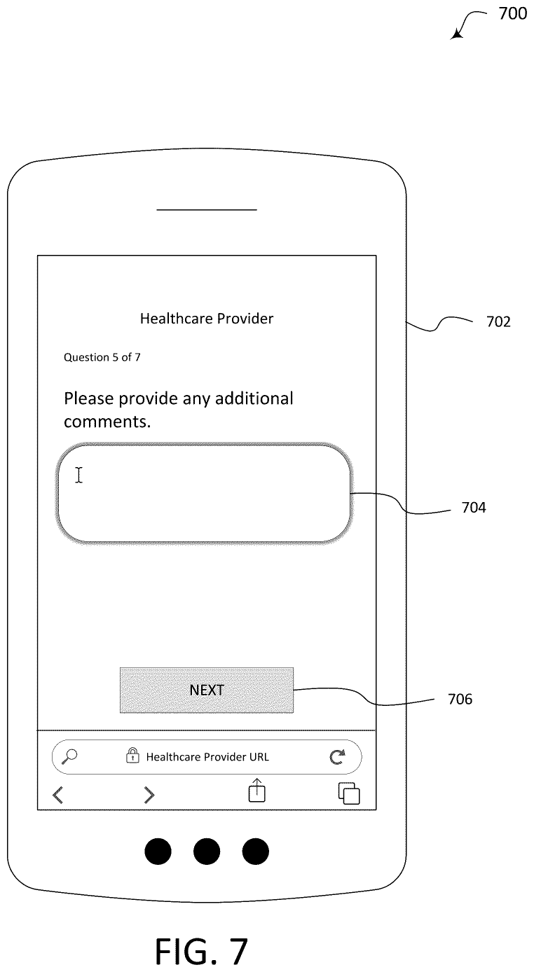

FIG. 7 depicts an example patient survey question page in a system 700 according to some embodiments of the disclosure. In some embodiments, the patient survey question page may be accessed on a patient device, such as phone 702, 602 of FIG. 6, 502 of FIG. 5, 402 of FIG. 4, 302 of FIG. 3, and/or patient devices 114 of FIG. 1. The patient survey page may include one or more patient survey questions, of many patient survey questions, such as depicted in FIG. 7. In some embodiments, patient survey questions may be answered with freeform text entered by the patient, as is depicted in FIG. 7. In some embodiments, the freeform text may be analyzed by the trust index system (e.g., 102 of FIG. 1) to identify trends and/or other factors affecting the trust index value of patients. The patient may go to a next patient survey question by clicking or selecting a button to advance, such as the “Next” button 706.

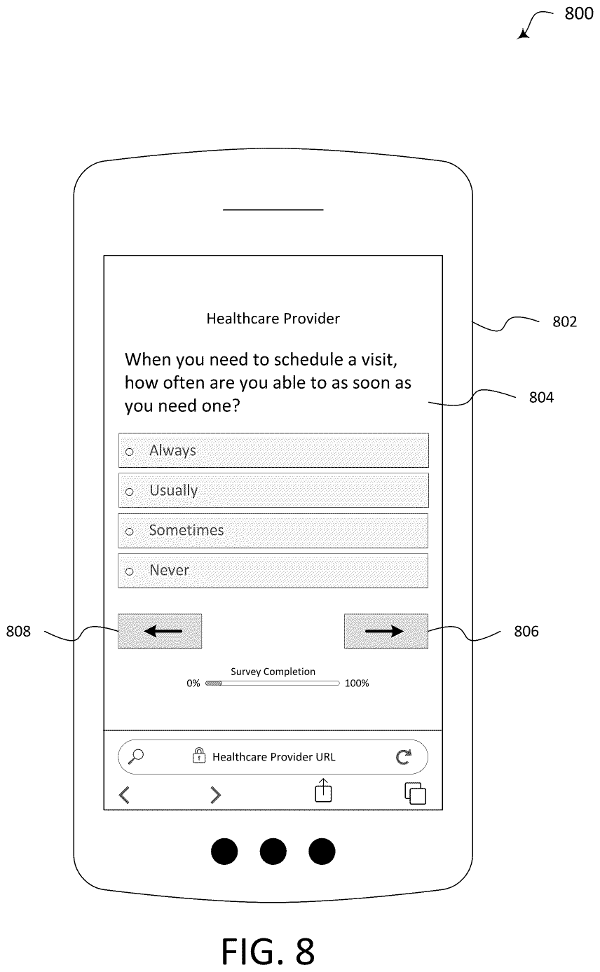

FIG. 8 depicts an example patient survey question page in a system 800 according to some embodiments of the disclosure. In some embodiments, the patient survey question page may be accessed on a patient device, such as phone 802, 702 of FIG. 7, 602 of FIG. 6, 502 of FIG. 5, 402 of FIG. 4, 302 of FIG. 3, and/or patient devices 114 of FIG. 1. The patient survey page may include one or more patient survey question 804, of many patient survey questions, such as is depicted in FIG. 8. For example, the patient survey question page indicates that patient survey question 804 is between 0% and 100% of “Survey Completion.” In other embodiments, there may be more or fewer patient survey questions. For example, the healthcare provider system (e.g., 104 of FIG. 1) may provide a different number of patient survey questions based on the type of patient interaction, such as treatment for an illness or a prescription refill. In some embodiments, more than one survey question may be displayed on a patient survey question page at a time. The trust index system (e.g., 102 of FIG. 1) may map the patient survey question 804 to one or more benchmark categories. In some embodiments, patient survey questions may be multiple choice, as is depicted in FIG. 8. For example, the patient may select an answer clicking or selecting on a touchscreen a radio button next to their desired response. In some embodiments, the patient may go to a previous patient survey question by selecting a button to go back, such as the back button 808. The patient may go to a next patient survey question or end the survey by clicking or selecting a button to advance, such as the next button 806.

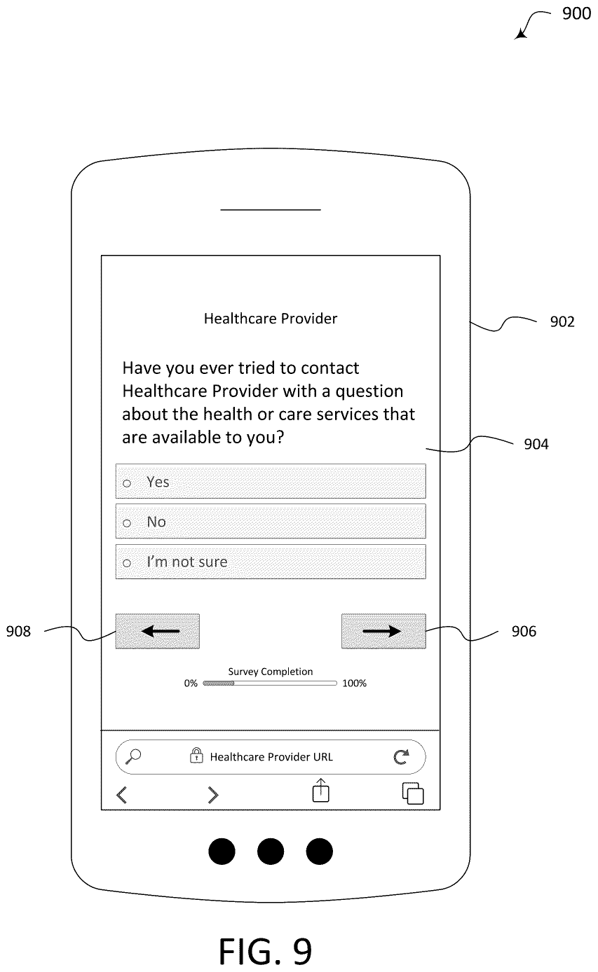

FIG. 9 depicts an example patient survey question page in a system 900 according to some embodiments of the disclosure. In some embodiments, the patient survey question page may be accessed on a patient device, such as phone 902, 802 of FIG. 8, 702 of FIG. 7, 602 of FIG. 6, 502 of FIG. 5, 402 of FIG. 4, 302 of FIG. 3, and/or patient devices 114 of FIG. 1. The patient survey page may include one or more patient survey questions 904, from many patient survey questions, such as is depicted in FIG. 9. For example, the patient survey question page indicates that patient survey question 904 is between 0% and 100% of “Survey Completion.” In other embodiments, there may be more or fewer patient survey questions. For example, the healthcare provider system (e.g., 104 of FIG. 1) may provide a different number of patient survey questions based on the type of patient interaction, such as treatment for an illness or a prescription refill. In some embodiments, more than one survey question may be displayed on a patient survey question page at a time. The trust index system (e.g., 102 of FIG. 1) may map the patient survey question 904 to one or more benchmark categories. In some embodiments, patient survey questions may be multiple choice, as is depicted in FIG. 9. For example, the patient may select an answer clicking or selecting on a touchscreen a radio button next to their desired response. In some embodiments, the patient may go to a previous patient survey question by selecting a button to go back, such as the back button 908. The patient may go to a next patient survey question or end the survey by clicking or selecting a button to advance, such as the next button 906.

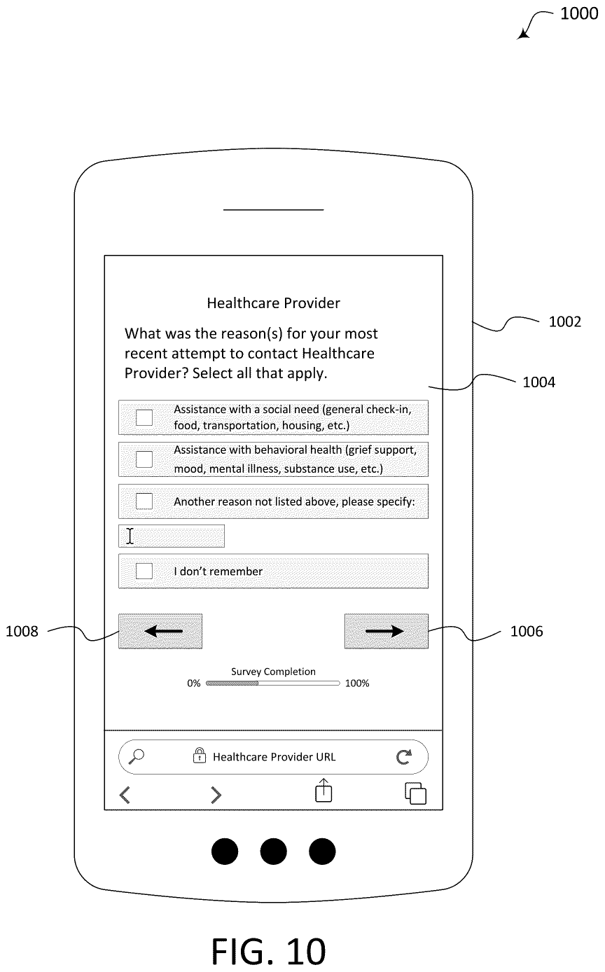

FIG. 10 depicts an example patient survey question page in a system 1000 according to some embodiments of the disclosure. In some embodiments, the patient survey question page may be accessed on a patient device, such as phone 1002, 902 of FIG. 9, 802 of FIG. 8, 702 of FIG. 7, 602 of FIG. 6, 502 of FIG. 5, 402 of FIG. 4, 302 of FIG. 3, and/or patient devices 114 of FIG. 1. The patient survey page may include one or more patient survey questions 1004, of many patient survey questions, such as is depicted in FIG. 10. For example, the patient survey question page indicates that patient survey question 1004 is between 0% and 100% of “Survey Completion.” In other embodiments, there may be more or fewer patient survey questions. For example, the healthcare provider system (e.g., 104 of FIG. 1) may provide a different number of patient survey questions based on the type of patient interaction, such as treatment for an illness or a prescription refill. In some embodiments, more than one survey question may be displayed on a patient survey question page at a time. The trust index system (e.g., 102 of FIG. 1) may map the patient survey question 1004 to one or more benchmark categories.

In some embodiments, patient survey questions may include one or more response types. For example, the patient survey question may be multiple choice, as is depicted in FIG. 10. For example, the patient may select an answer clicking or selecting on a touchscreen a checkbox next to their desired response. In some embodiments, the patient may select one or more than one response by selecting one or more checkboxes. In some embodiments, a response selection may include a text box. For example, by selecting the checkbox associated with a response that includes a text box, the patient may be able to enter freeform text into the text box using a keyboard. The freeform text may then be analyzed by the trust index system (e.g., 102 of FIG. 1). In some embodiments, the patient may go to a previous patient survey question by selecting a button to go back, such as the back button 1008. The patient may go to a next patient survey question or end the survey by clicking or selecting a button to advance, such as the next button 1006.

Although various specific examples of patient survey questions, and associated user interfaces, are illustrated and discussed above with reference to FIGS. 3-10, one of skill in the art will understand that the patient survey questions, the user interfaces, and the response mechanisms are not so limited. Purely by way of example, the systems and methods described above can utilize a wide variety of alternative questions, can modify the questions discussed above, and/or modify the response mechanism for any of the questions (e.g., to grade on a different scale, to receive comments in addition to a scale, to receive comments in place of a scale), and/or the like. Accordingly, the technology described herein is not limited to any specific example illustrated and discussed above.

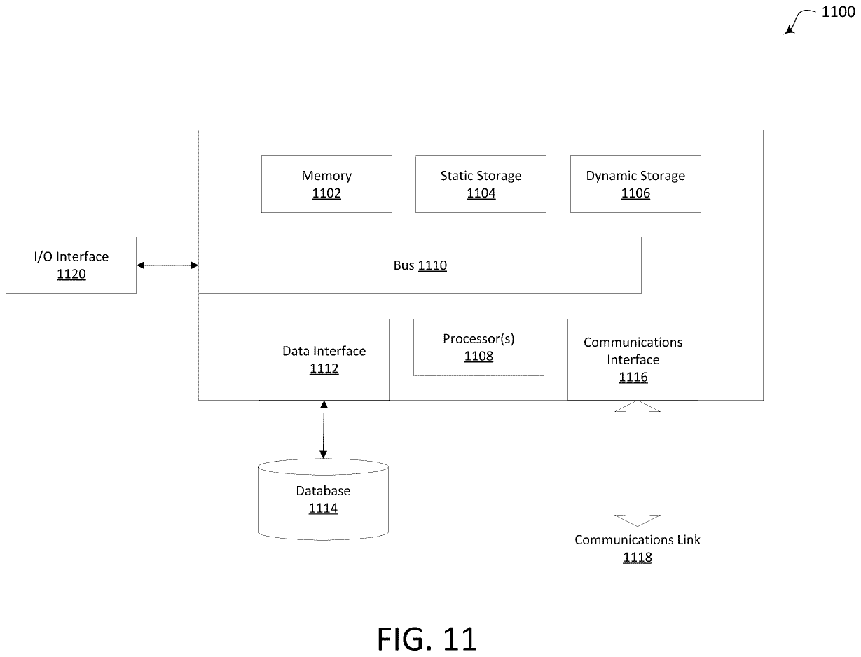

The trust index system (e.g., 102 of FIG. 1) may be implemented using various computing systems. Turning to FIG. 11, an example computing system 1100 may be used for implementing various embodiments in the examples described herein. In various embodiments, the patient devices 114 and/or the healthcare provider device 116 are also implemented by a computing system 1100. Various systems, such as healthcare provider systems 104, may be implemented by one or more computing systems 1100. This disclosure contemplates any suitable number of computing systems 1100. For example, the computing system 1100 may be a server, a desktop computing system, a mainframe, a mesh of computing systems, a laptop or notebook computing system, a tablet computing system, an embedded computer system, a system-on-chip, a single-board computing system, or a combination of two or more of these. Where appropriate, the computing system 1100 may include one or more computing systems; be unitary or distributed; span multiple locations; span multiple machines; span multiple data centers; or reside in a cloud, which may include one or more cloud components in one or more networks.

Computing system 1100 includes a bus 1110 (e.g., an address bus and a data bus) or other communication mechanism for communicating information, which interconnects subsystems and devices, such as processor 1108, memory 1102 (e.g., RAM), static storage 1104 (e.g., ROM), dynamic storage 1106 (e.g., magnetic or optical), communications interface 1116 (e.g., modem, Ethernet card, a network interface controller (NIC) or network adapter for communicating with an Ethernet or other wire-based network, a wireless NIC (WNIC) or wireless adapter for communicating with a wireless network, such as a WI-FI network), input/output (I/O) interface 1120 (e.g., keyboard, keypad, mouse, microphone). In particular embodiments, the computing system 1100 may include one or more of any such components.

In particular embodiments, processor 1108 includes hardware for executing instructions, such as those making up a computer program. The processor 1108 circuitry includes circuitry for performing various processing functions, such as executing specific software for performing specific calculations and tasks. In particular embodiments, I/O interface 1120 includes hardware, software, or both, providing one or more interfaces for communication between computing system 1100 and one or more I/O devices. Computing system 1100 may include one or more of these I/O devices, where appropriate. One or more of these I/O devices may enable communication between a person and computing system 1100.

In particular embodiments, communications interface 1116 includes hardware, software, or both providing one or more interfaces for communication (such as, for example, packet-based communication) between computing system 1100 and one or more other computer systems or one or more networks. One or more memory buses (which may each include an address bus and a data bus) may couple processor 1108 to memory 1102. Bus 1110 may include one or more memory buses, as described below. In particular embodiments, one or more memory management units (MMUs) reside between processor 1108 and memory 1102 and facilitate accesses to memory 1102 requested by processor 1108. In particular embodiments, bus 1110 includes hardware, software, or both coupling components of computing system 1100 to each other.

According to particular embodiments, computing system 1100 performs specific operations by processor 1108 executing one or more sequences of one or more instructions contained in memory 1102. For example, instructions for determining the trust index value and/or for the trust index model may be contained in memory 1102 and may be executed by the processor 1108. Such instructions may be read into memory 1102 from another computer readable/usable medium, such as static storage 1104 or dynamic storage 1106. In alternative embodiments, hard-wired circuitry may be used in place of or in combination with software instructions. Thus, particular embodiments are not limited to any specific combination of hardware circuitry and/or software. In various embodiments, the term “logic” means any combination of software or hardware that is used to implement all or part of particular embodiments disclosed herein.

The term “computer readable medium” or “computer usable medium” as used herein refers to any medium that participates in providing instructions to processor 808 for execution. Such a medium may take many forms including but not limited to, nonvolatile media and volatile media. Non-volatile media includes, for example, optical or magnetic disks, such as static storage 1104 or dynamic storage 1106. Volatile media includes dynamic memory, such as memory 1102.

Computing system 1100 may transmit and receive messages, data, and instructions, including program, e.g., application code, through communications link 1118 and communications interface 1116. Received program code may be executed by processor 1108 as it is received and/or stored in static storage 1104 or dynamic storage 1106 or other storage for later execution. A database 1114 may be used to store data accessible by the computing system 1100 by way of data interface 1112. For example, trust index values associated with patients, various patient survey questions, benchmark weights, and historical patient data may each be stored using a database 1114. In various examples, communications link 1118 may communicate with, for example, patient devices 114 and/or healthcare provider devices 116 to display user interfaces to the trust index system 102.

### EXAMPLES

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples can be combined in any suitable manner, and placed into a respective independent example. The other examples can be presented in a similar manner.

1. A computer implemented health services method comprising:
receiving a patient interaction notification for a patient from a healthcare provider for the patient;
transmitting a plurality of patient survey questions to a user device associated with the patient, wherein the plurality of patient survey questions based on a patient experience with the healthcare provider during the health event and comprise benchmark categories related to the healthcare experience;
receiving patient survey responses from the user device for the plurality of survey questions;
weighting the patient survey responses based on the benchmark categories;
   determining a patient trust index value for the patient using the weighted patient survey responses; and
outputting the patient trust index value to a provider device.

2. The method of example 1, further comprising triggering an alert based on the trust index value for the patient.

3. The method of example 2, wherein triggering the alert further comprises automatically transmitting a follow-up notification to the patient if the patient trust index value is below a threshold value.

4. The method of example 1, wherein the plurality of patient survey questions is tailored to the patient based on the patient interaction notification and health data associated with the patient.

5. The method of example 4, wherein the health data includes a patient health history.

6. The method of example 1, wherein the patient interaction notification is a discharge notification from a healthcare facility discharging the patient.

7. The method of example 1, further comprising generating a provider trust index value for the healthcare provider based on a plurality of patient trust index values.

8. The method of example 1, wherein the patient survey responses are received from an application programming interface (API) associated with the healthcare provider.

9. The method of example 1, wherein the benchmarks include at least one of satisfaction, individual trust, institutional trust, and post-appointment new patient satisfaction.

10. The method of example 1, generating an action item for the healthcare provider based on the patient trust index.

11. The method of example 10, wherein the action item for the healthcare provider is generated using a trust index model, wherein the trust index model is trained using a plurality of patient health data and a plurality of patient trust index values.

12. The method of example 11, wherein the action item for the healthcare provider comprises patient outreach based on the trust index value being below the threshold value.

13. The method of example 1, further comprising categorizing the trust index value by segment based on patient information.

14 The method of example 1, wherein the patient survey questions are displayed with visual indicators associated with the patient survey responses.

15. The method of example 14, wherein the visual indicators include color-coded buttons.

16. The method of example 14, wherein the visual indicators include color-coded face icons.

17. The method of example 1, wherein the patient survey questions are displayed with a textbox configured to receive freeform text input by the patient with a patient device.

18. A computer-implemented method for dynamically adjusting healthcare for a patient, the method comprising:
detecting a patient interaction with a healthcare provider;
selecting, from a plurality of patient survey questions, a subset of patient survey questions based on a category of the patient interaction and historical data associated with the patient, wherein each of the patient survey questions in the subset maps to one or more benchmark categories associated with trust between the patient and the healthcare provider;
providing the subset of patient survey questions to a patient user device associated with the patient;
receiving patient survey responses from the patient user device;
for each individual patient survey response, processing the individual patient survey response to weight and adjust the individual patient survey response based which of the one or more benchmark categories the individual patient survey response maps to;
calculating a trust index value using the weighted patient survey responses; and
determining one or more adjustments to protocols at the healthcare provider to improve trust from the patient.

19. The computer-implemented method of example 18 wherein processing the individual patient survey response to weight and adjust the individual patient survey response is further based on the historical data associated with the patient.

20. The computer-implemented method of example 19 wherein the historical data associated with the patient includes previous trust index values between the patient and the healthcare provider.

21. The computer-implemented method of example 19 wherein the healthcare provider is a first healthcare provider, and wherein the historical data associated with the patient includes previous trust index values between the patient and a second healthcare provider.

22. The computer-implemented method of example 18 wherein processing the individual patient survey response to weight and adjust the individual patient survey response is further based on historical data associated with the healthcare provider, and wherein the historical data associated with the healthcare provider includes an average trust index value for the healthcare provider.

23. The computer-implemented method of example 18 wherein calculating the trust index value is further based on the historical data associated with the patient, and wherein the previous trust index values between the patient and a plurality of healthcare providers.

24 The computer-implemented method of example 18 wherein the healthcare provider is associated with a category of healthcare, and wherein calculating the trust index value is further based on historical data associated with a plurality of healthcare providers within the category of healthcare.

25. The computer-implemented method of example 18 wherein determining the one or more adjustments to protocols at the healthcare provider comprises:
identifying, from a plurality of historical trust index values associated with a plurality of other patients, a subset of reference cases based on similarities between the patient and other patients in the subset of reference cases; and
comparing the calculated trust index value to the historical trust index values in the subset of reference cases.

26 The computer-implemented method of example 25 wherein the similarities between the patient and other patients in the subset of reference cases comprise similarities in demographic information between the patient and the other patients.

27. The computer-implemented method of example 25 wherein the similarities between the patient and other patients in the subset of reference cases comprise similarities healthcare treatments received.

28. A method for adjusting healthcare for a patient, the method comprising:
receiving a patient interaction notification for the patient from a healthcare provider system associated with a healthcare provider;
providing, to a user device associated with the patient, a set of patient survey questions, wherein the set of patient survey questions prompt the patient for inputs based on a patient experience with the healthcare provider during a healthcare interaction, and wherein each individual patient survey question in the set of patient survey questions maps to one or more benchmark categories;
receiving, from the user device, responses to the set of patient survey questions, wherein the responses comprise score ratings for one or more of the individual patient survey questions;
weighting the score ratings in the responses to the set of patient survey questions based at least partially on a mapping between the responses and the one or more benchmark categories;
determining one or more patient trust index values for the patient based at least partially on the weighted responses; and
determining, based at least partially on the one or more patient trust index values, one or more adjustments to operating protocols at the healthcare provider.

29. The method of example 28, wherein the benchmark categories comprise individual trust with regard to a specific healthcare provider, individual trust with regard to a specific team member of the specific healthcare provider, and institutional trust.

30. The method of example 28, wherein providing the plurality of patient survey questions comprises dynamically selecting the patient survey questions based on a type of the healthcare interaction and health data associated with the patient.

31. The method of example 30 wherein dynamically selecting the patient survey questions is further based on historical data associated with the patient, and wherein the historical data associated with the patient comprises previous responses to previous patient survey questions.

32. The method of example 28 wherein the plurality of patient survey questions comprise one or more freeform text entry prompts.

33. The method of example 32, further comprising, for each individual response to one of the one or more freeform text entry prompts, parsing the individual response using a natural language learning model to:
identify one or more benchmark categories associated with the individual response; and
assign scores to the individual response associated with each of the benchmark categories.

34. The method of example 33, wherein the natural language learning model is trained at least partially on historical responses to surveys that include both numerical response answers and freeform text answers.

35 The method of example 28 wherein the one or more adjustments to operating protocols at the healthcare provider include providing a follow-up notification to the patient, and wherein the method further comprises automatically sending the follow-up notification to the patient.

36. A computer-implemented method, comprising:
receiving a patient interaction notification from a healthcare provider system;
transmitting patient survey questions to a patient device, wherein the patient survey questions are tailored based on at least one of:
   a type of healthcare interaction;
   historical patient data; and
   institutional historical data;
receiving patient survey responses from the patient device;
adjusting the patient survey responses according to a plurality of benchmark categories associated with the patient survey questions, wherein adjusting the patient survey responses is based on the historical patient data;
determining a patient trust index value from the adjusted patient survey responses; and
outputting the patient trust index value to a healthcare provider device to enable adjustments to healthcare delivery at the healthcare provider.

37 The computer-implemented method of example 36 wherein at least a portion of the historical patient data includes previous trust index scores between the patient and one or more other healthcare providers.

### CONCLUSION

The technology described herein may be implemented as logical operations and/or modules in one or more systems. The logical operations may be implemented as a sequence of processor-implemented steps directed by software programs executing in one or more computer systems and as interconnected machine or circuit modules within one or more computer systems, or as a combination of both. Likewise, the descriptions of various component modules may be provided in terms of operations executed or effected by the modules. The resulting implementation is a matter of choice, dependent on the performance requirements of the underlying system implementing the described technology. Accordingly, the logical operations making up the embodiments of the technology described herein are referred to variously as operations, steps, objects, or modules. Furthermore, it should be understood that logical operations may be performed in any order, unless explicitly claimed otherwise or a specific order is inherently necessitated by the claim language.

In some implementations, articles of manufacture are provided as computer program products that cause the instantiation of operations on a computer system to implement the procedural operations. One implementation of a computer program product provides a non-transitory computer program storage medium readable by a computer system and encoding a computer program. It should further be understood that the described technology may be employed in special purpose devices independent of a personal computer.

The above specification, examples and data provide a complete description of the structure and use of exemplary embodiments of the invention as defined in the claims. Although various embodiments of the claimed invention have been described above with a certain degree of particularity, or with reference to one or more individual embodiments, it is appreciated that numerous alterations to the disclosed embodiments without departing from the spirit or scope of the claimed invention may be possible. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims. That is, from the foregoing, it will be appreciated that specific embodiments of the technology have been described herein for purposes of illustration, but well-known structures and functions have not been shown or described in detail to avoid unnecessarily obscuring the description of the embodiments of the technology. To the extent any material incorporated herein by reference conflicts with the present disclosure, the present disclosure controls.

Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded. Further, the terms “generally, “approximately,” and “about” are used herein to mean within at least within 10% of a given value or limit. Purely by way of example, an approximate ratio means within 10% of the given ratio.

Several implementations of the disclosed technology are described above in reference to the figures. The computing devices on which the described technology may be implemented can include one or more central processing units, memory, input devices (e.g., keyboard and pointing devices), output devices (e.g., display devices), storage devices (e.g., disk drives), and network devices (e.g., network interfaces). The memory and storage devices are computer-readable storage media that can store instructions that implement at least portions of the described technology. In addition, the data structures and message structures can be stored or transmitted via a data transmission medium, such as a signal on a communications link. Various communications links can be used, such as the Internet, a local area network, a wide area network, or a point-to-point dial-up connection. Thus, computer-readable media can comprise computer-readable storage media (e.g., “non-transitory” media) and computer-readable transmission media.

From the foregoing, it will also be appreciated that various modifications may be made without deviating from the disclosure or the technology. For example, one of ordinary skill in the art will understand that various components of the technology can be further divided into subcomponents, or that various components and functions of the technology may be combined and integrated. In addition, certain aspects of the technology described in the context of particular embodiments may also be combined or eliminated in other embodiments.

Furthermore, although advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A computer-implemented method for dynamically adjusting healthcare for a patient, the computer-implemented method comprising:
detecting a patient interaction with a healthcare provider;
selecting, from a plurality of patient survey questions, a subset of patient survey questions based on a category of the patient interaction and historical data associated with the patient, wherein each of the patient survey questions in the subset maps to one or more benchmark categories associated with trust between the patient and the healthcare provider;
providing the subset of patient survey questions to a patient user device associated with the patient;
receiving patient survey responses from the patient user device;
for each individual patient survey response, processing the individual patient survey response to weight and adjust the individual patient survey response based which of the one or more benchmark categories that the individual patient survey response maps to;
calculating a trust index value using the weighted patient survey responses; and
determining one or more adjustments to protocols at the healthcare provider to improve trust from the patient.

2. The computer-implemented method of claim 1 wherein processing the individual patient survey response is further based on the historical data associated with the patient.

3. The computer-implemented method of claim 2 wherein the historical data associated with the patient includes previous trust index values between the patient and the healthcare provider.

4. The computer-implemented method of claim 2 wherein the healthcare provider is a first healthcare provider, and wherein the historical data associated with the patient includes previous trust index values between the patient and a second healthcare provider.

5. The computer-implemented method of claim 1 wherein processing the individual patient survey response is further based on historical data associated with the healthcare provider, and wherein the historical data associated with the healthcare provider includes an average trust index value for the healthcare provider.

6. The computer-implemented method of claim 1 wherein calculating the trust index value is further based on the historical data associated with the patient, and wherein the historical data associated with the patient comprises previous trust index values between the patient and a plurality of healthcare providers.

7. The computer-implemented method of claim 1 wherein the healthcare provider is associated with a category of healthcare, and wherein calculating the trust index value is further based on historical data associated with a plurality of healthcare providers within the category of healthcare.

8. The computer-implemented method of claim 1 wherein determining the one or more adjustments to protocols at the healthcare provider comprises:
identifying, from a plurality of historical trust index values associated with a plurality of other patients, a subset of reference cases based on similarities between the patient and other patients in the subset of reference cases; and
comparing the calculated trust index value to the historical trust index values in the subset of reference cases.

9. The computer-implemented method of claim 8 wherein the similarities between the patient and other patients in the subset of reference cases comprise similarities in demographic information between the patient and the other patients.

10. The computer-implemented method of claim 8 wherein the similarities between the patient and other patients in the subset of reference cases comprise similarities healthcare treatments received.

11. A method for adjusting healthcare for a patient, the method comprising:
receiving a patient interaction notification for the patient from a healthcare provider system associated with a healthcare provider;
providing, to a user device associated with the patient, a set of patient survey questions, wherein the set of patient survey questions prompt the patient for inputs based on a patient experience with the healthcare provider during a healthcare interaction, and wherein each individual patient survey question in the set of patient survey questions maps to one or more benchmark categories;
receiving, from the user device, responses to the set of patient survey questions, wherein the responses comprise score ratings for one or more of the individual patient survey questions;
weighting the score ratings in the responses to the set of patient survey questions based at least partially on a mapping between the responses and the one or more benchmark categories;
determining one or more patient trust index values for the patient based at least partially on the weighted responses; and
determining, based at least partially on the one or more patient trust index values, one or more adjustments to operating protocols at the healthcare provider.

12. The method of claim 11, wherein the benchmark categories comprise individual trust with regard to a specific healthcare provider, individual trust with regard to a specific team member of the specific healthcare provider, and institutional trust.

13. The method of claim 11, wherein providing the set of patient survey questions comprises dynamically selecting the set of patient survey questions based on a type of healthcare interaction and health data associated with the patient.

14. The method of claim 13 wherein the health data associated with the patient comprises previous responses to previous patient survey questions and previous healthcare treatments.

15. The method of claim 11 wherein the set of patient survey questions comprises one or more freeform text entry prompts.

16. The method of claim 15, further comprising, for each individual response to one of the one or more freeform text entry prompts, parsing the individual response using a natural language learning model to:
identify one or more benchmark categories associated with the individual response; and
assign scores to the individual response associated with each of the benchmark categories.

17. The method of claim 16, wherein the natural language learning model is trained at least partially on historical responses to surveys that include both numerical response answers and freeform text answers.

18. The method of claim 11 wherein the one or more adjustments to operating protocols at the healthcare provider include providing a follow-up notification to the patient, and wherein the method further comprises automatically sending the follow-up notification to the patient.

19. A computer-implemented method, comprising:
receiving a patient interaction notification from a healthcare provider system;
transmitting patient survey questions to a patient device, wherein the patient survey questions are tailored based on at least one of:
a type of healthcare interaction;
historical patient data; and
institutional historical data;
receiving patient survey responses from the patient device;
adjusting the patient survey responses according to a plurality of benchmark categories associated with the patient survey questions, wherein adjusting the patient survey responses is based on the historical patient data;
determining a patient trust index value from the adjusted patient survey responses; and
outputting the patient trust index value to a healthcare provider device to enable adjustments to healthcare delivery at the healthcare provider.

20. The computer-implemented method of claim 19 wherein at least a portion of the historical patient data includes previous trust index scores between the patient and one or more other healthcare providers.
